(19) United States
(12) Patent Application Publication
(10) Pub. No.: US 2020/0015031 A1
(43) Pub. Date: Jan. 9, 2020

(54) LOCATION BASED METHOD OF OPERATING A MEDICAL DEVICE

(71) Applicant: General Electric Company, Schenectady, NY (US)

(72) Inventors: Rajeev Mandwal, Bangalore (IN); David Wayne Duckert, Wauwatosa, WI (US); Matthew Lane Pemberton, Hubertus, WI (US)

(73) Assignee: General Electric Company, Schenectady, NY (US)

(21) Appl. No.: 16/029,975

(22) Filed: Jul. 9, 2018

(51) Int. Cl. H04W 4/02; A61B 5/0402

(52) U.S. Cl. CPC H04W 4/02; H04W 84/12; A61B 5/0402

(57) ABSTRACT

A patient monitoring system that operates mobile medical devices based upon a known location of the mobile medical device within a facility. Each of the mobile medical devices communicates with a central control unit to obtain information and communicate information received from the patient. Each communication event between the mobile medical device and the central control unit can be used to identify the location of the mobile medical device within the facility. The known physical location of the mobile medical device can be correlated to one of a plurality of departments within the care facility. Once the location is correlated to the department within the care facility, the operation of the mobile medical device can be adjusted by selecting an operating mode and user interface based on the department. The automated adjustment of the operating mode and user interface improves the function of the medical device without requiring user input.

### BACKGROUND

The present disclosure generally relates to methods for operating medical devices. More specifically, the present disclosure relates to a method for operating a mobile medical device, such as a mobile ECG device, that utilizes the determined location of the medical device within a care facility to modify the operation of the medical device.

In the field of medicine, medical devices are often designed to be mobile such that they can be moved throughout a hospital or other healthcare facility such that a single medical device can be used in multiple locations with multiple different patients. Often, such medical devices are used in vastly different environments with vastly different patients, such as with infant patients, in emergency rooms, during surgery or in an intensive care unit. The medical device typically includes different modes of operation such that the operation of the medical device can be modified for use in the different units/wards within a hospital. When the medical device is going to be used with a specific patient, one of the selections made by a clinician is the mode of operation, which allows the operation of the medical device to be adjusted for use with the patient and the specific unit within the hospital. The setup process for the medical device is yet another operational step/procedure that the clinician must make prior to using the medical device with the patient.

### SUMMARY

This summary is provided to introduce a selection of concepts that are further described below in the Detailed Description. This summary is not intended to identify key or essential features of the claimed subject matter, nor is it intended to be used as an aid in limiting the scope of the claimed subject matter.

One embodiment of the present disclosure includes a method of operating a mobile medical device that is located within a care facility, such as a hospital. The method initially identifies the physical location of the medical device within the care facility. The determination of the physical location of the medical device can be done utilizing different location techniques, such as Wi-Fi triangulation or RFID sensing. Once the location of the medical device is known within the care facility, the determined location of the medical device is correlated with a map of the care facility. The physical location of the medical device can be shown and displayed on a map of the facility on a display at a central control unit or on a display of the mobile medical device. Once correlated with the map of the facility, the type of department of the determined location can be determined. As an example, the department type can be one of multiple different units within the facility or hospital, such as the emergency room, intensive care unit, pediatric wing, or other relevant locations where the medical device may be used. The map of the facility is created and stored within the medical device or at a central location that includes identifications of the different departments of the care facility such that the method can identify the category of department unit where the medical device is located.

Once the department is determined for the location of the medical device, one of a plurality of operating modes is selected for the medical device based upon the identified department category. The different operating modes are predetermined and include preferred operating parameters for each of the departments where the medical device may be located. Once the mode has been selected, the mobile medical device is operated using this selected mode such that the operation of the medical device is tailored to the department of the facility. The method further includes a mapping module that is able to access a map of the care facility and the system is able to display both the map of the care facility and the location of the medical device on a display of the medical device.

Another embodiment of the method of the present disclosure includes receiving an indication of a desired support function or peripheral device that may be needed by the clinician and is entered into the medical device by a clinician. The desired support function can include a need for charging, a need for printing or a need for a network interface. Once this need for a desired support function is received from the medical device, the method locates a peripheral device/support location that is capable of carrying out the desired support function based upon the determined physical location of the mobile medical device and the map of the care facility. Once the location of the peripheral device or support location is determined, this location is indicated on the display of the mobile medical device. The clinician can then move the mobile medical device to the shown location as needed.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 is a schematic diagram of an exemplary patient monitoring systems according to the present disclosure;

FIG. 2 depicts one embodiment of a medical device that can be used as part of the patient monitoring system;

FIG. 3A is a schematic illustration of a map of a care facility showing the location of a mobile medical device;

FIG. 3B is a schematic illustration of a map of a care facility showing the location of a mobile medical device after movement within the care facility;

FIG. 4A is a flowchart showing a sequence of operation according to one embodiment of the present disclosure; and

FIG. 4B is a continuation of the flowchart shown in FIG. 4A

### DETAILED DESCRIPTION

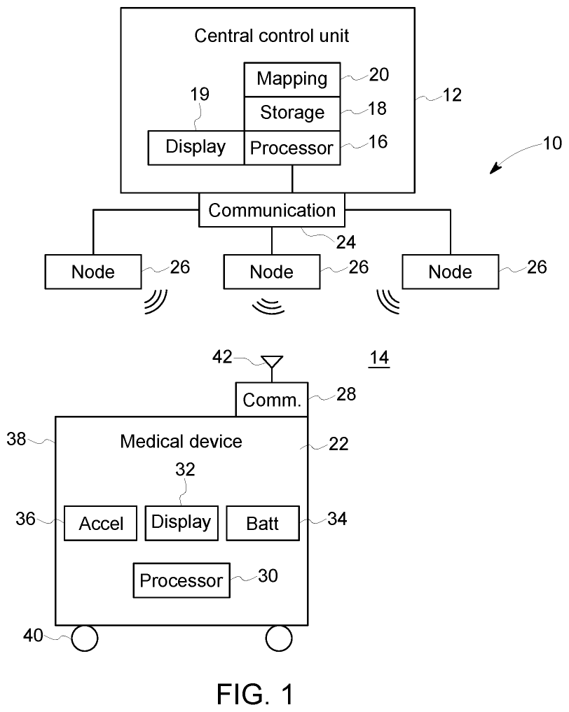

FIG. 1 illustrates an exemplary patient monitoring system 10 constructed in accordance with one embodiment of the present disclosure. In the embodiment shown in FIG. 1, a central control unit 12 is located within a care facility 14. In the embodiment shown, the central control unit 12 is a hospital information system (HIS) that is used to store patient records, monitor patients, clinicians and workflow within a hospital. In other contemplated embodiments, the central control unit 12 could be a central computing system that can be accessed throughout the care facility to retrieve information and to store information obtained from individual patients located throughout the care facility.

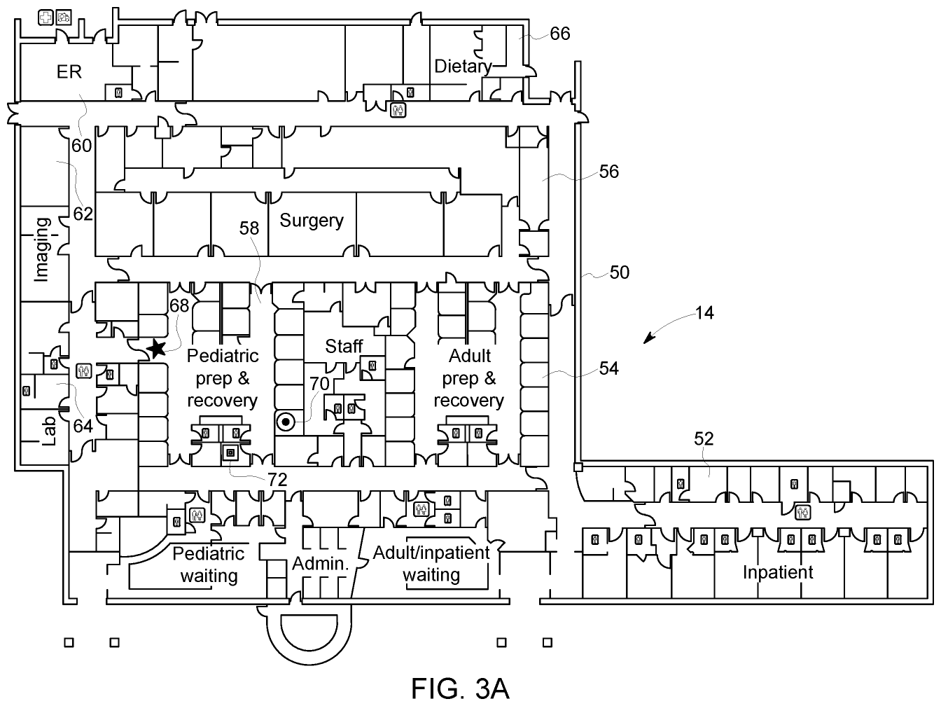

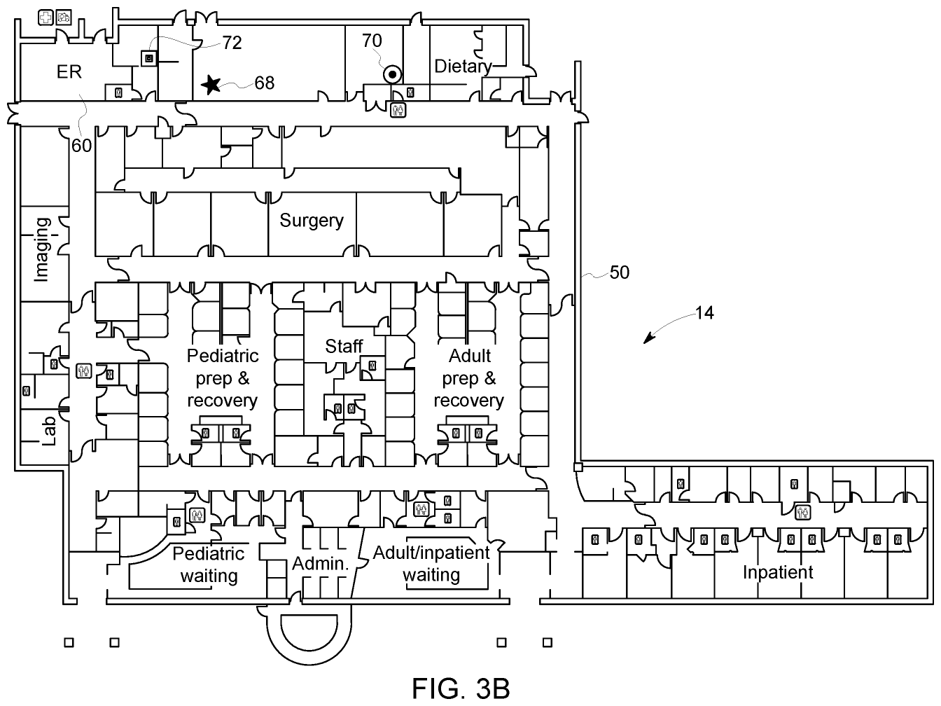

The central control unit 12 includes one or more processors 16 that control the operation of the system as will be described in greater detail below. The processor 16 communicates with a storage database 18 that can store received patient information and patient records. In the embodiment illustrated, the control unit 12 further includes a mapping module 20 that includes a detailed electronic map of the entire care facility 14 (as depicted, for example in FIG. 3A). The mapping module 20 is designed to receive an electronic map of the entire care facility, which can be obtained utilizing different mapping techniques, such as indoor GPS, Wi-Fi triangulation, cell tower triangulation or any other type or combination of technology that allows an accurate mapping of the care facility 14 to be created and stored electronically. The mapping module 20 provides an illustration of the layout of the care facility 14, but is also able to identify the location and department of a mobile medical device 22 with an acceptable degree of accuracy relative to the map of the care facility 14 in a manner that will be described in greater detail below.

In the embodiment shown in FIG. 1, the central control unit 12 includes a communication module 24 that controls communication throughout the facility 14. The communication module 24 in the embodiment shown is a Wi-Fi radio that includes a plurality of access nodes 26 distributed throughout the care facility 14. The plurality of nodes 26 provide for complete Wi-Fi coverage throughout the facility 14 such that wireless communication is supported throughout the entire facility 14. The Wi-Fi communication throughout the care facility 14 allows patient monitoring devices, computer access terminals, nursing stations and other devices to communicate directly to the control unit 12 utilizing wireless communication techniques, such as Wi-Fi. The use of the Wi-Fi radio and associated nodes 26 is a commonly used communication system within a care facility to communicate information from the central control unit 12 to individual access points located throughout the care facility.

The patient monitoring system 10 shown in FIG. 1 includes a plurality of mobile medical devices 22. The mobile medical device 22 is designed to be moved throughout the care facility such that each mobile medial device 22 can be used at multiple locations with multiple different patients. Although the mobile medical device 22 can have many different configurations, one contemplated configuration is a mobile ECG monitoring device. The mobile ECG monitoring device can be moved throughout the care facility and used in various different departments of the care facility. As an example, the mobile medical device 22 can be moved to an emergency room, intensive care unit, pediatric wing or any other relevant department within the care facility where an ECG monitoring device may be needed. Since the mobile medical device 22 can be moved throughout the care facility, it is desirable for the mobile medical device 22 to know its location within the care facility 14 so that the operation of the device can be optimized based on the location.

The mobile medical device 22 shown in FIG. 1 can be a variety of different devices, such as an ECG monitor, a blood pressure monitor, a patient monitor including a variety of sensors or any other type of medical device that could be mobile and moved throughout the care facility. Each mobile medical device will typically include common features, such as a communication module 28, a processor 30, a display 32 and/or a battery power supply 34. In the embodiment shown, the mobile medical device 22 further includes an accelerometer 36, although such component is not necessarily required within the mobile medical device 22. In the embodiment shown, the mobile medical device includes a support cart 38 having a plurality of wheels that allows the mobile medical device 22 to be easily transported throughout the care facility. However, if the mobile medical device 22 was of a small enough size, the mobile medical device 22 could be carried throughout the care facility while operating within the scope of the present disclosure.

The communication module 28 shown in FIG. 1 includes an antenna 42 that allows the mobile medical device 22 to communicate utilizing wireless communication techniques. In the embodiment shown, the communication module 28 is a Wi-Fi radio that communicates utilizing conventional Wi-Fi communication techniques. However, other communication techniques, such as but not limited to Bluetooth and GPRS, are contemplated as being within the scope of the present disclosure.

As can be understood in FIG. 1, as the mobile medical device 22 moves throughout the care facility, the communication between the mobile medical device 22 and the central control unit 12 takes place utilizing one or more of the communication nodes 26. One method of locating the device 22 includes identifying the Wi-Fi signature from the nodes 26 and finding the location of the nodes 26. As part of the mapping module 20, the central control unit can place each of the nodes 26 on the electronic representation of the care facility. By utilizing Wi-Fi triangulation, the processor 16 of the central control unit 12 is able to determine the precise location of the mobile medical device 22 within the care facility. Based upon the known location of the mobile medical device 22, the operation of the mobile medical device 22 can be adjusted to optimize the performance of the mobile medical device 22 based upon its known location.

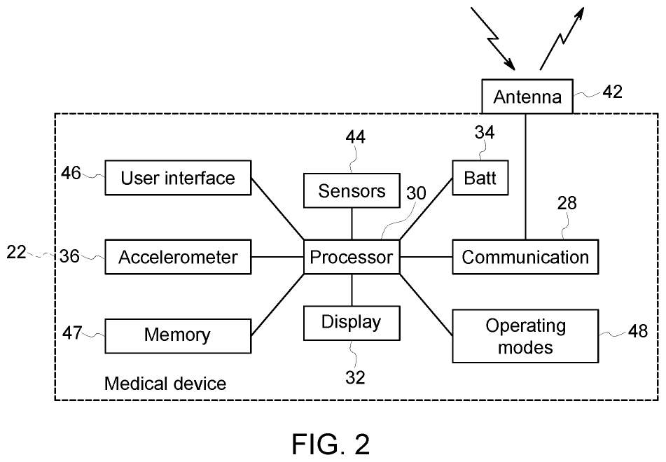

Referring now to FIG. 2, a detailed schematic illustration of the operating components of a representative mobile medical device 22 is shown. As described previously, the mobile medical device 22 includes the processor 30. As illustrated in FIG. 2, the processor 30 communicates with the sensors 44 to obtain information from the patient. In an embodiment in which the mobile medical device 22 is an ECG monitor, the sensors 44 are conventional ECG sensors that are attached to the patient to receive ECG information from the patient. The processor 30 is coupled to the communication module 28 such that the mobile medical device 22 can communicate to the central control unit of the care facility utilizing wireless communication techniques. In the embodiment shown, the communication module 28 is a Wi-Fi radio that communicates utilizing Wi-Fi communication techniques utilizing the antenna 42. In this manner, the mobile medical device 22 is able to not only relay information obtained from the patient but can also receive instructions and information from the central control unit.

In the embodiment shown in FIG. 2, the mobile medical device 22 includes the display 32 that can be used to provide information to a clinician relative to the information obtained from the sensors 44 or from the central control unit. In many embodiments, the display 32 is a touchscreen that also serves as the user interface for a clinician to enter information and control operation of the mobile medical device 22. The mobile medical device 22 includes a user interface module 46. The user interface module 46 includes a series of different graphical user interfaces (GUIs) stored in memory locations 47 that can be retrieved by the processor 30 and used to control the operation of the display 32. The graphic user interfaces stored in the user interface module 46 allow the processor 30 to configure the display 32 including different input buttons, display formats and to control the type of information displayed to the clinician on the display 32.

In addition to the user interface module 46, the mobile medical device 22 also includes an operating mode module 48. The operating mode module 48 includes a series of stored commands and operating parameters that adjust the operation of the mobile medical device 22 based upon either manual selections made by the clinician or automatically based upon the known location of the mobile medical device within the care facility in accordance with the present disclosure.

As an illustrative example, the operating mode module 48 may include multiple different modes of operating the mobile medical device based upon the category of the department within the care facility where the mobile medical device 22 is located. In a first illustrative example, the operating modes may include an Emergency Room Mode, a Pediatric Mode, a Surgery Mode, and a Stress Testing Mode. Each of these individual operating modes, when selected, causes the processor 30 to operate in a slightly different manner and may use a different graphical user interface retrieved from the user interface module.

In one illustrative example, when the ECG monitoring device is in the Emergency Room Mode, the processor 30 will retrieve the most recent ECG for the patient and show the most recent ECG on the display 32. The display of the most recent ECG on the display 32 allows the clinician to immediately assess the recent history of the patient without having to utilize the graphical user interface to retrieve such information. In addition, in the Emergency Room Mode, the graphical user interface of the display 32 is changed to be greatly simplified to aid in use in what may be a hectic emergency room environment. As an example, the patient date of birth, patient ID and order number could be removed from the graphical user interface in the Emergency Room Mode to reduce clutter on the display.

As a further illustrative example, if the ECG device is changed to the Pediatric Mode, alarm limits and normal measurement parameters are adjusted since an infant patient will have different results as compared to an adult patient. Other parameters, such as alarm limits for heartrate, can also be automatically set in the Pediatric Mode since a normal infant heartrate is much different from an adult heartrate.

In a Stress Test Mode, an ECG monitoring device will also adjust the heartrate and normal ECG alarms and parameters since the patient is being tested and the information obtained by the sensors 44 will be different from normal resting conditions.

Other operating modes are certainly contemplated as being within the scope of the present disclosure. Further, when the mobile medical device 22 is a medical device other than an ECG monitor, the operating modes will be specific to the type of mobile medical device. However, the operating modes stored in the operating mode module 48 are typically based upon either the type of patient or the type of care area or department within the care facility.

As discussed previously, the central control unit 12 shown in FIG. 1 includes a communication module 24 having multiple communication nodes 26. The multiple communication nodes 26 are distributed throughout the care facility and are able to communicate with each of the mobile medical device 22. As part of this communication process, the processor 16 is able to determine the location of the mobile medical device 22 utilizing Wi-Fi triangulation techniques. In a healthcare facility 14 that includes a significant number of nodes 26, the Wi-Fi triangulation technique is able to accurately locate the mobile medical device 22 within the facility by comparing the Wi-Fi signatures of the communication messages to the known locations of the communication nodes 26 relative to a map of the healthcare facility.

FIG. 3A illustrates a representative map 50 of a care facility that is stored within the mapping module 20 of the central control unit 12. The map 50 shown in FIG. 3A includes the different departments of the care facility with identified categories of the individual department. In the representative illustration of FIG. 3A, the care facility includes an inpatient department 52, an adult care department 54, a surgery department 56, a pediatric department 58, an emergency room department 60, an imaging department 62, a lab department 64 and a dietary department 66. Although a variety of different departments within the care facility are shown in FIG. 3A, it should be understood that other categories or departments within the care facility could be included in different types of care facilities. The illustration in FIG. 3A is meant to show one sample layout of a care facility that includes a plurality of different departments each of which have a different category.

As described previously, through Wi-Fi triangulation or other wireless communication location techniques, the central control unit 12 is able to determine the location of the mobile medical device 22 as the mobile medical device 22 moves throughout the care facility. Once the central control unit determines the location of the mobile medical device, the central control unit is able to provide a visual indicator 68 of the device on the map 50. In the embodiment shown in FIG. 3A, the visual indicator 68 is a star and shows that the mobile medical device is located within the pediatric department 58. In addition to the visual indicator 68 showing the location of the mobile medical device, the central control unit is able to provide a visual indicator 70 showing the nearest charging station and a visual indicator 72 showing the nearest location of a printer. The visual representation of the map 50 including both the visual indicator 68 showing the location of the mobile medical device, the visual indicator 70 showing the location of a charging station and the visual indicator 72 showing the location of a printer can be shown on either the display 19 of the central control unit or on the display 32 of the mobile medical device.

When the map and location of the mobile medical device are shown on the display 32 of the mobile medical device 22, the operator is more easily able to determine the location within the care facility and is able to identify the location of relevant peripheral devices or support functions, such as the location of a charging station or printer. In the embodiment shown in FIG. 3A, the nearest location of both the charging station and printer are automatically shown to the operator. However, these indicators could be displayed based upon a request from the operator rather than automatically shown on the map 50.

FIG. 3B illustrates the location of the mobile medical device as the mobile medical device moves throughout the facility. In the embodiment of FIG. 3B, the indicator 68 shows that the mobile medical device has been moved to the emergency room department 60. Once again, the nearest location of the charging station 70 and the printer 72 are shown on the map 50. Thus, as the mobile medical device is moved throughout the care facility, the central control unit can determine the location of the mobile medical device, identify the category of the department within the care facility where the mobile medical device is located and provide information to the mobile medical device such that the display 32 of the mobile medical device can show this information to the operator.

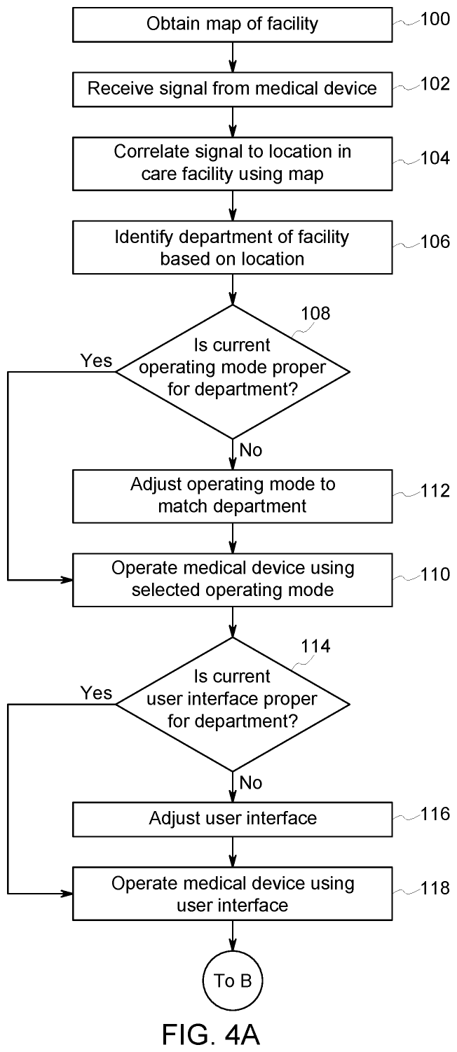

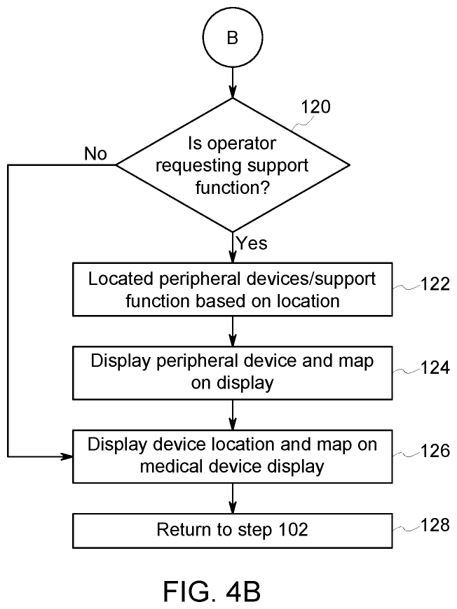

FIG. 4A illustrates one method of operating the mobile medical device shown and described in the previous figures and description. Although FIGS. 4A and 4B illustrate one possible method of operating a mobile medical device and patient monitoring system, it is contemplated that other methods are possible and included within the scope of the present disclosure.

In step 100, the central control unit of the care facility obtains a map of the facility. The step of obtaining the map can be done by either mapping the facility utilizing a Wi-Fi transceiver and moving throughout the facility to obtain Wi-Fi signatures from the various nodes distributed throughout the different departments within the care facility or by obtaining such a representation of the care facility from an outside provider. Typically, the map of the facility will include not only the visual representation of the facility and the various departments, but also representative Wi-Fi signatures for locations throughout the facility. The map of the facility obtained in step 100 is stored within the mapping module 20 of the central control unit 12, as illustrated in FIG. 1.

Once the map of the facility has been obtained and stored in the mapping module, the central control unit 12 operates the communication module 24 and various nodes 26 to receive wireless communication signals from the mobile medical device, as illustrated in step 102. The signals from the mobile medical device received in step 102 can either be commands from the mobile medical device, information obtained from the sensors associated with the mobile medical device or regular communication messages sent at defined intervals. Since the mobile medical device is movable throughout the care facility, the received signals from the medical device, whether the signals include measurement data or are regular test signals, allow the central control unit to determine the location of the mobile medical device at each communication.

After receiving a signal from the mobile medical device, the processor of the central control unit correlates the signal received from the medical device to a location within the care facility using the map stored in the mapping module 20, as set forth in step 104. As discussed previously, the central control unit includes a mapping module that includes a full map of the care facility along with a table or database of Wi-Fi triangulation results, which allows the system to correlate the signal received from the mobile medical device to a location within the care facility.

After the location of the mobile medical device is correlated to the map of the care facility, the system identifies the department of the care facility where the mobile medical device is located, as indicated in step 106. The departments of the care facility fall into common categories, such as surgery, pediatric ward, adult care unit, emergency room or any other category of the different departments located within the care facility. Once the category of the department within the facility is identified, the system proceeds to step 108 in which this information is sent to the mobile medical device.

When this information is received by the mobile medical device, the processor 30 contained within the mobile medical device 22 determines whether the current operating mode of the mobile medical device matches the identified department. As an example, if the current operating mode is the Emergency Room Mode and the system determines that the mobile medical device is located in the emergency room department 60, the mobile medical device continues to operate in the current mode, as indicated by the combination of steps 108 and 110. However, if the processor 30 of the mobile medical device 22 determines that the current operating mode does not match the department category, the processor 30 accesses the proper operating mode from the operating mode module 48. As an example, if the identified department category for the mobile medical device indicates that the mobile medical device is in the pediatric ward but the mode of operation is the Emergency Room Mode, the system adjusts the operating mode to the Pediatric Mode in step 112. In this manner, the processor 30 is able to select the correct operating mode based upon the identified location of the mobile medical device within the healthcare facility. This selection of the proper operating mode is done automatically and does not require any input from an operator. Once the correct operating mode is selected, the medical device is operated using the selected operating mode, as indicated in step 110. Although the operating mode is initially selected automatically based on the location of the medical device, this selection could be manually overridden by an operator using the user interface on the medical device.

Once the proper operating mode is selected, the method moves to step 114 where the processor determines whether the current user interface is proper for the department category. As with the operating mode, the processor 30 determines whether the selected user interface matches the department category. As an example, if the current user interface is one for the emergency room and the location of the mobile medical device is the pediatric unit, the system proceeds to step 116 where the user interface is adjusted. The user interface is adjusted in step 116 by having the processor 30 access the user interface module 46 and select the proper user interface for the department category. In step 118, the medical device is then operated using the selected user interface or, if the current user interface was proper, the medical device continues to operate using this user interface.

As can be understood by the steps in FIG. 4A, the location of the mobile medical device is determined by the central control unit and based upon this location, the processor of the mobile medical device 22 selects the correct operating mode and user interface. Once these two selections are made, the mobile medical device begins to operate utilizing the selected user interface and operating mode without requiring intervention by the operator. The proper selection of the user interface and operating mode allows the mobile medical device to be automatically adjusted based upon a known location of the mobile medical device, which will save the operator time in configuring the mobile medical device.

Referring now to FIG. 4B, the method further includes the step 120 of allowing an operator to request a support function or a peripheral device. As an example, the operator may need to recharge the mobile medical device and thus needs to locate a suitable charging station. Alternatively, the operator may indicate a desire to print results from the mobile medical device and needs to know the location of the nearest printer. If the operator indicates such need for a support function or peripheral device in step 112, the method moves to step 122 where the processor of the central control unit identifies the nearest or most efficient location for the desired peripheral device or support function based upon the known location of the mobile medical device. In step 124, the central control unit relays the mapping information and location information to the mobile medical device where the location of the mobile medical device, peripheral device and map can be shown on the display 32. FIGS. 3A and 3B show the type of display that could be shown to an operator on the display of the mobile medical device. The displays of FIGS. 3A and 3B show the layout of the care facility, the location of the mobile medical device as well as the location of a charging station 70 and printer 72.

If the operator did not request any support function or peripheral device, the system moves to step 126 where only the map and medical device location are shown on the display of the medical device. In this manner, the operator of the mobile medical device can learn the location within the hospital floor and identify a direction of movement required to move between different departments within the care facility. Once the device displays the location and map, the system returns to step 102 and receives the next signal from the medical device such that the system continues to monitor the location of the device within the facility.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of operating a mobile medical device located within a facility, comprising the steps of:
creating a map of the facility that includes a plurality of departments defined within the map of the facility;
determining the physical location of the medical device within the facility;
identifying a current department for the medical device based on the determined location of the medical device relative to the the map of the facility;
selecting one of a plurality of operating modes based on the identified current department; and
operating the mobile medical device in the selected operating mode.

2. The method of claim 1 wherein the medical device is an ECG monitor.

3. (canceled)

4. The method of claim 1 wherein the physical location of the medical device is determined using a wireless location technique.

5. The method of claim 4 wherein the mobile medical device includes a Wi-Fi radio.

6. The method of claim 1 further comprising the step of displaying the map of the facility and the determined physical location of the medical device on a display of the medical device.

7. The method of claim 1 further comprising the step of adjusting a user interface of the medical device based on the department of the determined location.

8. A method of operating a mobile medical device located within a facility, comprising the steps of:
creating a map of the facility that includes a plurality of departments defined within the map of the facility;
determining a physical location of the medical device within the facility;
correlating the determined location of the medical device to the map of the facility to identify the department where the medical device is located;
receiving an indication of a desired support function from the medical device;
locating a peripheral device within the facility capable of carrying out the desired support function based on the determined physical location; and
displaying the map of the facility and the determined physical location of the medical device and a location of the peripheral device on a display of the medical device.

9. (canceled)

10. (canceled)

11. The method of claim 8 wherein the desired support function includes charging the medical device and printing from the medical device.

12. A method of operating a mobile medical device located within a facility, comprising the steps of:
creating a map of the facility that includes a plurality of departments defined within the map of the facility;
determining a physical location of the medical device within the facility;
identifying a current department for the medical device based on the determined location of the medical device and the map of the facility;
selecting one of a plurality of operating modes based on the identified current department;
operating the mobile medical device in the selected operating mode; and
adjusting a user interface of the medical device based on the identified current department of the facility.

13. The method of claim 12 wherein the medical device is an ECG monitor.

14. The method of claim 12 wherein the physical location of the medical device is determined using a wireless location technique.

15. The method of claim 14 wherein the mobile medical device includes a Wi-Fi radio.

16. The method of claim 12 further comprising the step of displaying the map of the facility and the determined physical location of the medical device on a display of the medical device.

17. The method of claim 12 further comprising the steps of:
receiving an indication of a desired support function from the medical device;
locating a peripheral device capable of carrying out the desired support function based on the determined physical location; and
indicating the location of the peripheral device.

18. The method of claim 17 further comprising the step of displaying the location of the peripheral device with the map of the facility on a display of the medical device.

19. The method of claim 17 wherein the desired support function includes charging the medical device and printing from the medical device.
